(19) United States
(12) Patent Application Publication
(10) Pub. No.: US 2022/0354884 A1
(43) Pub. Date: Nov. 10, 2022

(54) MUSCLE FATIGUE RECOVERY PROMOTING AGENT AND METHOD FOR PRODUCING MUSCLE FATIGUE RECOVERY-PROMOTING LIQUID

(71) Applicant: NIPPON EKITAN Corporation, Minato-ku, Tokyo (JP)

(72) Inventors: Yoshihiko SUGIHARA, Tokyo (JP); Hiroyuki MURAKAMI, Tokyo (JP); Takahiro EGUCHI, Tokyo (JP)

(73) Assignee: NIPPON EKITAN Corporation, Minato-ku, Tokyo (JP)

(21) Appl. No.: 17/622,525

(22) PCT Filed: Jun. 26, 2020
(86) PCT No.: PCT/JP2020/025159

§ 371 Date: Dec. 23, 2021

(30) Foreign Application Priority Data
Jun. 28, 2019 (JP) 2019-122078

(51) Int. Cl. A61K 33/00; A61K 9/08; A61P 21/00

(52) U.S. Cl. CPC A61K 33/00; A61K 9/08; A61P 21/00

(57) ABSTRACT

An object of the present invention is to provide a muscle fatigue recovery-promoting agent that can effectively promote the recovery of muscle fatigue, a method for producing a muscle fatigue recovery-promoting liquid, comprising the step of contacting the muscle fatigue recovery-promoting agent with a liquid, etc. In the present invention, ice (preferably CO₂ hydrate) having a CO₂-content rate of 3% by weight or more is used. The ice (preferably CO₂ hydrate) having a CO₂-content rate of 3% by weight or more or melted water thereof can be applied to the skin at the site of a targeted muscle to thereby promote the recovery of fatigue of the muscle.

### TECHNICAL FIELD

The present invention relates to a muscle fatigue recovery-promoting agent and a method for producing a muscle fatigue recovery-promoting liquid, and more specifically relates to a muscle fatigue recovery-promoting agent for preparing before use a muscle fatigue recovery-promoting liquid for application to the skin, a method for producing the muscle fatigue recovery-promoting liquid, etc.

### BACKGROUND ART

Muscle fatigue is a generic name for muscle pain resulting from heavy exercise, tired arms or legs resulting from sudden exercise, and shoulder stiffness, lower back pain, etc. caused by continuing to have a given posture for a long time. Many factors are known to participate in the muscle fatigue. Examples of such a factor include (1) intracellular accumulation of metabolic by-products (H+, inorganic phosphoric acid and ammonia, etc.), (2) decline in Ca2⁺ releasing function in sarcoplasmic reticulum, (3) lack of ATP necessary for muscle contraction, (4) depletion of energy substances such as muscle glycogen and liver glycogen, and (5) injury to muscles.

A general method for improving muscle fatigue involves waiting for spontaneous recovery. For example, a method of applying a commercially available anti-inflammatory agent, analgesic, or the like to an affected part, a method of massaging an affected part, and a method of orally ingesting a composition containing a substance improving muscle fatigue are known as methods for promoting fatigue recovery when muscle fatigue has severe symptoms. As for the aforementioned composition containing a substance improving muscle fatigue, for example, patent document 1 discloses a muscle fatigue improving agent containing alanylglutamine or a salt thereof as an active ingredient, and patent document 2 discloses an amino acid-containing composition for muscle fatigue recovery promotion containing 9 types of amino acids such as leucine at a particular ratio. Also, patent document 3 discloses a blood circulation promoting agent for external use containing carbonated water, wherein the agent is applied, together with ultrasonic irradiation, to a target site of a living body. In patent document 3, a method for artificially dissolving carbonic acid in water includes a chemical method of injecting a tablet or the like containing baking soda to water, a method of mixing carbonic acid with water, followed by dissolution under pressure, a method using a static mixer, a method using a multilayer composite hollow fiber membrane, and a method of disintegrating and dissolving bubbles. However, these methods require a special apparatus such as an ultrasonic apparatus and are therefore insufficient, for example, because there is a limitation on environments where the methods can be carried out and the recovery of muscle fatigue may not be sufficiently obtained. Hence, there has still been a demand for a novel muscle fatigue recovery-promoting agent.

A substance called CO₂ hydrate (carbon dioxide hydrate) is known as one type of ice having a high CO₂-content rate. The CO₂ hydrate refers to a clathrate compound in which a carbon dioxide molecule is confined in the vacant space of crystals of a water molecule. The water molecule constituting crystals is called “host molecule”, and the molecule confined in the vacant space of crystals of the water molecule is called “guest molecule” or “guest substance”. The CO₂ hydrate is degraded into CO₂ (carbon dioxide) and water when melted and therefore generates CO₂ upon melting. The CO₂ hydrate can be produced by placing CO₂ and water under conditions of a low temperature and a high CO₂ partial pressure and can be produced, for example, under conditions involving a certain temperature and a higher CO₂ partial pressure than the equilibrium pressure of the CO₂ hydrate at the temperature (hereinafter, also referred to as “CO₂ hydrate formation conditions”). The CO₂-content rate of the CO₂ hydrate can be on the order of approximately 3 to 28% by weight, though differing depending on a method for producing the CO₂ hydrate, and is much higher than the CO₂-content rate of carbonated water (approximately 0.5% by weight).

The addition or mixing of CO₂ hydrate into beverages is known as a purpose of the CO₂ hydrate. For example, patent document 4 discloses that a carbonated beverage is produced by mixing CO₂ hydrate into a beverage and thereby imparting carbonic acid to the beverage. Patent document 5 discloses that a lukewarm beverage is cooled while a vapid beverage is replenished with carbon dioxide gas, by adding a carbonic acid replenishment medium formed by covering CO₂ hydrate with ice to the beverage. Also, patent document 6 discloses a method for refrigerating any one subject to be refrigerated selected from a fresh food, a dairy product, Japanese cake and a fresh flower using CO₂ hydrate, comprising housing the CO₂ hydrate and the subject to be refrigerated without contact therebetween in a hermetically sealable container. Furthermore, patent document 7 discloses a cooling apparatus that can achieve the prevention of rush of blood to head or a pleasant bathing environment by cooling a body site of a bather, a beverage for a bather, etc., using oxygen hydrate (O₂ hydrate).

However, it has not previously been known that the recovery of muscle fatigue can be promoted by applying ice, such as CO₂ hydrate, having CO₂-content rate of 3% by weight or more to the skin.

### PRIOR ART DOCUMENTS

### Patent Documents

Patent document 1: WO 2007/108530
Patent document 2: WO 2013/021891
Patent document 3: WO 2014/208723
Patent document 4: Japanese unexamined Patent Application Publication No. 2005-224146
Patent document 5: Japanese Patent No. 4969683
Patent document 6: Japanese Patent No. 4500566
Patent document 7: Japanese unexamined Patent Application Publication No. 2007-319280

### SUMMARY OF THE INVENTION

### Object to be Solved by the Invention

An object of the present invention is to provide a muscle fatigue recovery-promoting agent that can effectively promote the recovery of muscle fatigue, a method for producing a muscle fatigue recovery-promoting liquid, comprising the step of contacting the muscle fatigue recovery-promoting agent with a liquid or melting the muscle fatigue recovery-promoting agent as it is, etc.

### Means to Solve the Object

The present inventors have conducted diligent studies to attain the object and consequently completed the present invention by finding that when ice (preferably CO₂ hydrate) having a CO₂-content rate of 3% by weight or more or melted water thereof is applied to the skin of the body of an animal, the recovery of fatigue of a muscle present beneath the skin can be effectively promoted.

The present inventors previously found that when the body is cooled with ice having a CO₂-content rate of 3% by weight or more, the body can be cooled with the blood flow volume of the skin controlled and a conventional icing method can be improved, and thus filed a patent application (Japanese Patent Application No. 2018-200952). However, this application dose not disclose that ice having a CO₂-content rate of 3% by weight or more actually promotes the recovery of muscle fatigue and particularly, promotes the recovery of induced muscle strength upon electrical stimulation.

Specifically, the present invention relates to:

(1) a muscle fatigue recovery-promoting agent comprising ice having a CO₂-content rate of 3% by weight or more;
(2) the muscle fatigue recovery-promoting agent according to the above (1), wherein the ice having a CO₂-content rate of 3% by weight or more is CO₂ hydrate;
(3) the muscle fatigue recovery-promoting agent according to the above (1) or (2), wherein the muscle fatigue recovery-promoting agent is an agent for promoting recovery for induced muscle strength upon electrical stimulation after exercise;
(4) the muscle fatigue recovery-promoting agent according to any one of the above (1) to (3), wherein the ice having a CO₂-content rate of 3% by weight or more is ice having a size of 3 mm or larger in terms of a maximum length and having a CO₂-content rate of 3% by weight or more;
(5) the muscle fatigue recovery-promoting agent according to any one of the above (1) to (4), wherein the ice having a CO₂-content rate of 3% by weight or more is consolidated CO₂ hydrate;
(6) the muscle fatigue recovery-promoting agent according to any one of the above (1) to (5), wherein the muscle fatigue recovery-promoting agent is used to prepare before use a muscle fatigue recovery-promoting liquid for application to the skin; and
(7) a method for producing a muscle fatigue recovery-promoting liquid for application to the skin, comprising the step of contacting a muscle fatigue recovery-promoting agent according to any one of the above (1) to (6) with a liquid or melting the muscle fatigue recovery-promoting agent as it is.

### Effect of the Invention

The present invention can provide a muscle fatigue recovery-promoting agent that can effectively promote the recovery of muscle fatigue, a method for producing a muscle fatigue recovery-promoting liquid, comprising the step of contacting the muscle fatigue recovery-promoting agent with a liquid or melting the muscle fatigue recovery-promoting agent as it is, etc.

### BRIEF DESCRIPTION OF DRAWING

FIG. 1 shows a diagram illustrating results of measuring the induced muscle strength of triceps surae muscle upon electrical stimulation in test 2 in Examples mentioned later. In FIG. 1, “Before fatigue” depicts results of measuring the induced muscle strength of triceps surae muscle upon electrical stimulation of a test subject before execution of a fatigue task, and “20 min after fatigue” depicts results of measuring the induced muscle strength of triceps surae muscle upon electrical stimulation of the test subject after a lapse of 20 minutes after execution of the fatigue task. These measurement results are indicated by relative values (%) when the average induced muscle strength of each group before fatigue is defined as 100%. In FIG. 1, the rhombic marker depicts results about an ice group, the rectangular marker depicts results about a CO₂ hydrate group, and the triangular marker depicts results about a noncontact group (group using neither ice nor CO₂ hydrate). For the ice group, the skin at the site of triceps surae muscle of each test subject was contacted with ice via gauze for 20 minutes after execution of the fatigue task. For the CO₂ hydrate group, the skin at the site of triceps surae muscle of each test subject was contacted with CO₂ hydrate via gauze for 20 minutes after execution of the fatigue task.

### MODE OF CARRYING OUT THE INVENTION

The present invention includes embodiments such as

[1] a muscle fatigue recovery-promoting agent comprising ice having a CO₂-content rate of 3% by weight or more (hereinafter, also referred to as “CO₂-rich ice”) (hereinafter, also referred to as the “muscle fatigue recovery-promoting agent of the present invention”); and
[2] a method for producing a muscle fatigue recovery-promoting liquid for application to the skin, comprising the step of contacting the muscle fatigue recovery-promoting agent of the present invention with a liquid or melting the muscle fatigue recovery-promoting agent of the present invention as it is (hereinafter, also referred to as “the producing method of the present invention”).

In the present specification, the “agent” can be restated into a “substance” or a “composition”. The present specification also describes, for example, a substance for muscle fatigue recovery promotion and a composition for muscle fatigue recovery promotion.

The present invention also includes the following aspects:

[3] a muscle fatigue recovery-promoting liquid for application to the skin, the liquid comprising 200 ppm or more of carbonic acid and containing 5 million or more ultrafine bubbles/mL (hereinafter, also referred to as the “muscle fatigue recovery-promoting liquid of the present invention”);
[4] a method for promoting the recovery of muscle fatigue in an animal, comprising the step of applying CO₂-rich ice (preferably CO₂ hydrate) or the muscle fatigue recovery-promoting agent or the muscle fatigue recovery-promoting liquid of the present invention to the systemic or local skin of the animal (hereinafter, also referred to as the “muscle fatigue recovery promotion method of the present invention”);
[5] use of CO₂-rich ice (preferably CO₂ hydrate) or the muscle fatigue recovery-promoting agent or the muscle fatigue recovery-promoting liquid of the present invention for promoting the recovery of muscle fatigue (preferably for promoting the recovery of induced muscle strength upon electrical stimulation) in an animal;
[6] a method for using CO₂-rich ice (preferably CO₂ hydrate) or the muscle fatigue recovery-promoting agent or the muscle fatigue recovery-promoting liquid of the present invention for promoting the recovery of muscle fatigue (preferably for promoting the recovery of induced muscle strength upon electrical stimulation) in an animal; and
[7] use of CO₂-rich ice (preferably CO₂ hydrate) in the production of the muscle fatigue recovery-promoting agent or the muscle fatigue recovery-promoting liquid of the present invention.

### 1. <Muscle Fatigue Recovery-Promoting Agent of Present Invention>

The muscle fatigue recovery-promoting agent of the present invention is not particularly limited as long as the muscle fatigue recovery-promoting agent contains ice having a CO₂-content rate of 3% by weight or more (“CO₂-rich ice”). Although the mechanism of action under which the muscle fatigue recovery-promoting agent of the present invention exerts a muscle fatigue recovery promoting effect is not clear, it is considered that physiological action ascribable to the transdermal absorption of a high concentration of CO₂ derived from the CO₂-rich ice is related thereto because the muscle fatigue recovery-promoting agent has a higher muscle fatigue recovery promoting effect than that of ice.

### (Ice Having CO₂-Content Rate of 3% by Weight or More)

The CO₂-rich ice according to the present invention may be CO₂-rich ice other than CO₂ hydrate and is preferably CO₂ hydrate, more preferably consolidated CO₂ hydrate, from the viewpoint of obtaining a higher muscle fatigue recovery promoting effect (preferably recovery promoting effect on induced muscle strength upon electrical stimulation). As for the CO₂-rich ice according to the present invention, CO₂-rich ice other than CO₂ hydrate may be used without the use of CO₂ hydrate, CO₂ hydrate may be used without the use of CO₂-rich ice other than CO₂ hydrate, or CO₂-rich ice other than CO₂ hydrate and CO₂ hydrate may be used in combination. Also, as for the CO₂ hydrate, unconsolidated CO₂ hydrate may be used without the use of consolidated CO₂ hydrate, consolidated CO₂ hydrate may be used without the use of unconsolidated CO₂ hydrate, or unconsolidated CO₂ hydrate and consolidated CO₂ hydrate may be used in combination.

The CO₂ hydrate is a solid clathrate compound in which a carbon dioxide molecule is confined in the vacant space of crystals of a water molecule. The CO₂ hydrate is usually ice-like crystals and, when left, for example, under a standard atmospheric pressure condition and a temperature condition that melts ice, releases CO₂ while melted. As mentioned above, the CO₂-rich ice used in the present invention is preferably CO₂ hydrate rather than CO₂-rich ice other than CO₂ hydrate, more preferably consolidated CO₂ hydrate. This is because the muscle fatigue recovery-promoting agent of the present invention can produce a higher concentration of CO₂ bubbles (preferably ultrafine bubbles) upon contact with a liquid and as a result, produces a higher muscle fatigue recovery promoting effect (preferably recovery promoting effect on induced muscle strength upon electrical stimulation). The “ultrafine bubbles” are microscopic bubbles having a diameter of 1000 nm or smaller in a solvent such as water under ordinary pressure. The ultrafine bubbles have excellent properties such as (1) a significantly large bubble interface surface area, (2) a large intra-bubble pressure, (3) high gas dissolution efficiency, and (4) a slow bubble ascension rate, as compared with usual bubbles having a diameter of 1 mm or larger. Although an ultrafine bubble generation apparatus is usually essential for the formation of the ultrafine bubbles, use of the CO₂-rich ice (preferably CO₂ hydrate, more preferably consolidated CO₂ hydrate) enables CO₂ microscopic bubbles (preferably ultrafine bubbles) to be conveniently formed without the use of an ultrafine bubble generation apparatus.

The CO₂-rich ice (preferably CO₂ hydrate) according to the present invention is not particularly limited by the degree of a concentration (the number of ultrafine bubbles/mL) of ultrafine bubbles that can be generated in water in ice water when the CO₂-rich ice is added to water. In the case of adding 300 mg of the CO₂-rich ice according to the present invention per mL of water, preferred examples of such CO₂-rich ice can include CO₂-rich ice that can generate preferably 5 million or more ultrafine bubbles/mL, more preferably 10 million or more ultrafine bubbles/mL, further preferably 20 million or more ultrafine bubbles/mL, more preferably 25 million or more ultrafine bubbles/mL, further preferably 30 million or more ultrafine bubbles/mL, more preferably 35 million or more ultrafine bubbles/mL, further preferably 50 million or more ultrafine bubbles/mL, more preferably 75 million or more ultrafine bubbles/mL, further preferably 1 hundred million or more ultrafine bubbles/mL, more preferably 150 million or more ultrafine bubbles/mL, further preferably 2 hundred million or more ultrafine bubbles/mL, more preferably 250 million or more ultrafine bubbles/mL (the ultrafine bubbles are preferably CO₂ ultrafine bubbles) in water in terms of the concentration (the number of ultrafine bubbles/mL) of the ultrafine bubbles (preferably CO₂ ultrafine bubbles) in water in the ice water.

In the present specification, the value of the concentration (the number of ultrafine bubbles/mL) of the ultrafine bubbles in water may be a measurement value of any measurement method that can measure the concentration of the ultrafine bubbles, and is preferably a measurement value according to the following measurement method R, more preferably a measurement value according to the following measurement method R1.

### (Measurement Method R)

The concentration (the number of ultrafine bubbles/mL) of the ultrafine bubbles in water is measured by a laser diffraction/scattering method (preferably quantitative laser diffraction/scattering method) or a nanotracking method.

### (Measurement Method R1)

Ice of −80 to 0° C. having a CO₂-content rate of 3% by weight or more is added at 300 mg/mL to water of 25° C. to prepare ice water of 0 to 2° C. containing ice having a CO₂-content rate of 3% by weight or more. Then, the concentration (the number of ultrafine bubbles/mL) of the ultrafine bubbles in water in the ice water is measured by a laser diffraction/scattering method (preferably quantitative laser diffraction/scattering method) or a nanotracking method.

In the present specification, preferred examples of the measurement of the concentration of the ultrafine bubbles by the laser diffraction/scattering method include the measurement of the concentration of the ultrafine bubbles using SALD-7500 ultrafine bubble size analysis system manufactured by Shimadzu Corp. The SALD-7500 ultrafine bubble size analysis system is a measurement apparatus based on the quantitative laser diffraction/scattering method. In the present specification, preferred examples of the measurement of the concentration of the ultrafine bubbles by the nanotracking method include the measurement of the concentration of the ultrafine bubbles using Nanosight NS300 manufactured by Malvern Panalytical Ltd.

Examples of the upper limit of the concentration of the ultrafine bubbles (preferably CO₂ ultrafine bubbles) that can be generated by the CO₂-rich ice according to the present invention in water include, but are not particularly limited to, an ultrafine bubble concentration of 10 billion or less ultrafine bubbles/mL or 1 billion or less ultrafine bubbles/mL.

More specific examples of the concentration of the ultrafine bubbles (preferably CO₂ ultrafine bubbles) that can be generated by the CO₂-rich ice according to the present invention in water include 5 million to 10 billion ultrafine bubbles/mL, 5 million to 1 billion ultrafine bubbles/mL, 10 million to 10 billion ultrafine bubbles/mL, 10 million to 1 billion ultrafine bubbles/mL, 20 million to 10 billion ultrafine bubbles/mL, 20 million to 1 billion ultrafine bubbles/mL, 25 million to 10 billion ultrafine bubbles/mL, 25 million to 1 billion ultrafine bubbles/mL, 30 million to 10 billion ultrafine bubbles/mL, 30 million to 1 billion ultrafine bubbles/mL, 35 million to 10 billion ultrafine bubbles/mL, 35 million to 1 billion ultrafine bubbles/mL, 50 million to 10 billion ultrafine bubbles/mL, 50 million to 1 billion ultrafine bubbles/mL, 75 million to 10 billion ultrafine bubbles/mL, 75 million to 1 billion ultrafine bubbles/mL, 1 hundred million to 10 billion ultrafine bubbles/mL, 1 hundred million to 1 billion ultrafine bubbles/mL, 150 million to 10 billion ultrafine bubbles/mL, 150 million to 1 billion ultrafine bubbles/mL, 2 hundred million to 10 billion ultrafine bubbles/mL, 2 hundred million to 1 billion ultrafine bubbles/mL, 250 million to 10 billion ultrafine bubbles/mL, and 250 million to 1 billion ultrafine bubbles/mL.

The CO₂-content rate of the CO₂-rich ice (preferably CO₂ hydrate) according to the present invention is not particularly limited as long as the CO₂-content rate is 3% by weight or more. The CO₂-content rate is preferably 5% by weight or more, more preferably 7% by weight or more, further preferably 10% by weight or more, more preferably 13% by weight or more, further preferably 16% by weight or more, more preferably 18% by weight or more, from the viewpoint of obtaining a higher concentration of CO₂ bubbles (preferably ultrafine bubbles) and obtaining a higher muscle fatigue recovery promoting effect (preferably recovery promoting effect on induced muscle strength upon electrical stimulation). Examples of the upper limit value include, but are not particularly limited to, 30% by weight, 28% by weight, 26% by weight, and 24% by weight. More specific examples of the CO₂-content rate of the CO₂-rich ice (preferably CO₂ hydrate) include 5 to 30% by weight, 7 to 30% by weight, 10 to 30% by weight, 13 to 30% by weight, 16 to 30% by weight, 18 to 30% by weight, 5 to 28% by weight, 7 to 28% by weight, 10 to 28% by weight, 13 to 28% by weight, 16 to 28% by weight, 18 to 28% by weight, 5 to 26% by weight, 7 to 26% by weight, 10 to 26% by weight, 13 to 26% by weight, 16 to 26% by weight, and 18 to 26% by weight.

The CO₂-content rate of the CO₂-rich ice according to the present invention can be adjusted depending on, for example, a “higher or lower CO₂ partial pressure” in producing the CO₂-rich ice according to the present invention. For example, the CO₂-content rate of the CO₂-rich ice can be elevated by elevating the CO₂ partial pressure. When the CO₂-rich ice is CO₂ hydrate, the CO₂-content rate of the CO₂ hydrate can be adjusted depending on, for example, a “higher or lower CO₂ partial pressure”, the “degree of dewatering treatment”, the “presence or absence of compression treatment”, and/or a “higher or lower compression pressure of compression treatment” in producing the CO₂ hydrate. For example, the CO₂-content rate of the CO₂ hydrate can be elevated by “elevating the CO₂ partial pressure”, “increasing the degree of dewatering treatment”, “performing compression treatment”, and/or “elevating the consolidation pressure of compression treatment” in producing the CO₂ hydrate. The CO₂-rich ice such as CO₂ hydrate releases CO₂ contained in the CO₂-rich ice such as CO₂ hydrate when melted, and the weight is decreased by the release. Therefore, the CO₂-content rate of the CO₂-rich ice such as CO₂ hydrate can be calculated, for example, from change in weight in melting the CO₂-rich ice such as CO₂ hydrate at ordinary temperature according to the following equation (1):

(CO₂-content rate)=(Sample weight before melting−Sample weight after melting)/Sample weight before melting) Equation (1)

It is preferred that every CO₂-rich ice (preferably CO₂ hydrate) contained in the muscle fatigue recovery-promoting agent of the present invention should have a CO₂-content rate of 3% by weight or more. However, the muscle fatigue recovery-promoting agent may contain ice or CO₂ hydrate having a CO₂-content rate of less than 3% by weight within a range that produces the effect of the present invention (muscle fatigue recovery promoting effect, preferably recovery promoting effect on induced muscle strength upon electrical stimulation). The ratio (% by weight) of the ice or the CO₂ hydrate having a CO₂-content rate of less than 3% by weight to the CO₂-rich ice (preferably CO₂ hydrate) contained in the muscle fatigue recovery-promoting agent of the present invention is 10% by weight or less, preferably 5% by weight or less, more preferably 3% by weight or less, further preferably 1% by weight or less.

The shape of the CO₂-rich ice (preferably CO₂ hydrate) according to the present invention can be appropriately set. Examples thereof include: a substantially spherical shape; a substantially ellipsoidal shape; a substantially polyhedral shape such as a substantially cuboid shape; and a shape further having irregularities in these shapes. The CO₂-rich ice (preferably CO₂ hydrate) according to the present invention may be a fragment (mass) in various shapes obtained by appropriately crushing a mass of the CO₂-rich ice (preferably CO₂ hydrate).

The size of the CO₂-rich ice (preferably CO₂ hydrate) according to the present invention is not particularly limited and can be appropriately set. The lower limit of the maximum length of the CO₂-rich ice (preferably CO₂ hydrate) according to the present invention is preferably 3 mm or larger, more preferably 5 mm or larger, further preferably 7 mm or larger, more preferably 10 mm or larger. Examples of the upper limit of the maximum length include 150 mm or smaller, 100 mm or smaller, 80 mm or smaller, and 60 mm or smaller. More specific examples of the maximum length include 3 mm or larger and 150 mm or smaller, 3 mm or larger and 100 mm or smaller, 3 mm or larger and 80 mm or smaller, 3 mm or larger and 60 mm or smaller, 5 mm or larger and 150 mm or smaller, 5 mm or larger and 100 mm or smaller, 5 mm or larger and 80 mm or smaller, 5 mm or larger and 60 mm or smaller, 10 mm or larger and 150 mm or smaller, 10 mm or larger and 100 mm or smaller, 10 mm or larger and 80 mm or smaller, and 10 mm or larger and 60 mm or smaller.

In the present specification, the “maximum length of the CO₂-rich ice” means the length of the longest line segment among line segments that connect two points on the surface of a mass of the CO₂-rich ice and pass through the gravity center of the mass. When the CO₂-rich ice has, for example, a substantially ellipsoidal shape, the maximum length represents a major axis (longest diameter). In the case of a substantially spherical shape, the maximum length represents a diameter. In the case of a substantially cuboid shape, the maximum length represents the length of the longest diagonal among diagonals. In the present specification, the “minimum length of the CO₂-rich ice” means the length of the shortest line segment among line segments that connect two points on the surface of a mass of the CO₂-rich ice (preferably CO₂ hydrate) and pass through the gravity center of the mass. The maximum length or the minimum length may be measured using a commercially available image analysis-type particle size distribution measurement apparatus or the like or may be measured by placing a ruler on a mass of the CO₂-rich ice (preferably CO₂ hydrate).

A preferred form of the CO₂-rich ice (preferably CO₂ hydrate) according to the present invention is CO₂-rich ice (preferably CO₂ hydrate) having an aspect ratio (maximum length/minimum length) preferably within the range of 1 to 5, more preferably within the range of 1 to 4, further preferably within the range of 1 to 3.

The size of the CO₂-rich ice (preferably CO₂ hydrate) can be adjusted by the following methods. For example, the maximum length of CO₂-rich ice other than CO₂ hydrate can be adjusted by adjusting the maximum length of a mold for producing the CO₂-rich ice and/or adjusting the degree of crush in crushing the CO₂-rich ice after production. The maximum length of CO₂ hydrate can be adjusted by adjusting the maximum length of a mold for use in the compression molding of the CO₂ hydrate and/or adjusting the degree of crush in crushing the CO₂ hydrate after compression molding. The minimum length can be adjusted by adjusting the minimum length of a mold and/or adjusting the degree of crush in crushing the CO₂-rich ice after production.

The method for producing the CO₂-rich ice according to the present invention is not particularly limited as long as the method can produce the CO₂-rich ice. Examples of the method for producing CO₂-rich ice other than CO₂ hydrate include a method of freezing raw material water while blowing CO₂ into the raw material water under conditions that do not meet CO₂ hydrate formation conditions. A conventional method such as a gas-liquid stirring technique of stirring raw material water while blowing CO₂ into the raw material water under conditions that meet CO₂ hydrate formation conditions, or a water spray technique of spraying raw material water into CO₂ under conditions that meet CO₂ hydrate formation conditions can be used as a method for producing CO₂ hydrate. CO₂ hydrate formed by such a technique usually contains fine particles of the CO₂ hydrate in a slurry state mixed with unreacted water. Therefore, it is preferred to perform dewatering treatment for elevating the concentration of the CO₂ hydrate. It is preferred that CO₂ hydrate having a relatively low water-content rate by the dewatering treatment (i.e., a relatively high concentration of CO₂ hydrate) should be compression-molded into a given shape (e.g., a spherical shape or a cuboid shape) in a pellet molding machine. The compression-molded CO₂ hydrate can be preferably used as one type of consolidated CO₂ hydrate according to the present invention. The compression-molded CO₂ hydrate may be used as it is in the present invention or may be further crushed, if necessary, for example. As mentioned above, a method using raw material water is relatively widely used as a method for producing CO₂ hydrate. However, a method for producing CO₂ hydrate may be used which involves reacting fine ice (raw material ice) instead of water (raw material water) with CO₂ under conditions of a low temperature and a low CO₂ partial pressure.

The “CO₂ hydrate formation conditions” described above are conditions involving a higher CO₂ partial pressure (CO₂ pressure) than the equilibrium pressure of CO₂ hydrate at the temperature, as mentioned above. The “conditions involving a higher CO₂ partial pressure than the equilibrium pressure of CO₂ hydrate” described above are indicated by conditions involving a combination of a CO₂ pressure and a temperature within a region on a high pressure side (e.g., an upper region in a CO₂ hydrate equilibrium pressure curve wherein the ordinate depicts a CO₂ pressure and the abscissa depicts a temperature) in a CO₂ hydrate equilibrium pressure curve (e.g., the ordinate depicts a CO₂ pressure and the abscissa depicts a temperature) disclosed in FIG. 2 of J. Chem. Eng. Data (1991) 36, 68-71 or FIG. 7 or 15 of J. Chem. Eng. Data (2008), 53, 2182-2188. Specific examples of the CO₂ hydrate formation conditions include conditions involving a combination of a “temperature within the range of −20 to 4° C.” and a “carbon dioxide pressure within the range of 1.8 to 4 MPa”, and conditions involving a combination of a “temperature within the range of −20 to −4° C.” and a “carbon dioxide pressure within the range of 1.3 to 1.8 MPa”.

The content of the CO₂-rich ice (preferably CO₂ hydrate) in the muscle fatigue recovery-promoting agent according to the present invention is not particularly limited and can be, for example, within the range of 5 to 100% by weight, preferably within the range of 30 to 100% by weight, more preferably within the range of 50 to 100% by weight, further preferably within the range of 70 to 100% by weight.

In the present invention, the “consolidated CO₂ hydrate” means CO₂ hydrate having a CO₂ hydrate ratio of 40 to 90% (preferably 50 to 90%, more preferably 60 to 90%, further preferably 70 to 90%). The CO₂ hydrate ratio means the ratio (%) of the weight of the CO₂ hydrate to the weight of a mass of the CO₂ hydrate. The CO₂ hydrate ratio can be calculated according to the following equation (2):

CO₂ hydrate ratio (%)={(Sample weight before melting −Sample weight after melting)+(Sample weight before melting −Sample weight after melting)/44×5.75×18}×100/Sample weight before melting Equation (2)

The equation (2) will be described below. (Sample weight before melting −Sample weight after melting) represents the weight of CO₂ gas occupying cages. The amount of water necessary for the clathration of CO₂ gas as hydrate is calculated using a theoretical hydration number of 5.75, a CO₂ molecular weight of 44, and a water molecular weight of 18, and the other water molecules are regarded as adhesion water that does not constitute the hydrate.

Preferred consolidated CO₂ hydrate according to the present invention is not particularly limited by the degree of a concentration (the number of ultrafine bubbles/mL) of ultrafine bubbles that can be generated in water in ice water when the consolidated CO₂ hydrate is added to water. In the case of adding 300 mg of the consolidated CO₂ hydrate according to the present invention per mL of water, examples of such consolidated CO₂ hydrate include CO₂ hydrate that can generate 50 million or more ultrafine bubbles/mL, more preferably 75 million or more ultrafine bubbles/mL, further preferably 1 hundred million or more ultrafine bubbles/mL, more preferably 150 million or more ultrafine bubbles/mL, further preferably 2 hundred million or more ultrafine bubbles/mL, more preferably 250 million or more ultrafine bubbles/mL in water in terms of the concentration (the number of ultrafine bubbles/mL) of the ultrafine bubbles (preferably CO₂ ultrafine bubbles) in water in the ice water. Preferred consolidated CO₂ hydrate according to the present invention is not particularly limited by the degree of a concentration (the number of ultrafine bubbles/mL) of ultrafine bubbles that can be generated in melted water obtained by melting the consolidated CO₂ hydrate as it is. In the case of melting the consolidated CO₂ hydrate as it is, examples of such consolidated CO₂ hydrate include CO₂ hydrate that can generate 1 hundred million or more ultrafine bubbles/mL, more preferably 2 hundred million or more ultrafine bubbles/mL, further preferably 3 hundred million or more ultrafine bubbles/mL, more preferably 5 hundred million or more ultrafine bubbles/mL, further preferably 7 hundred million or more ultrafine bubbles/mL, more preferably 1 billion or more ultrafine bubbles/mL in melted water in terms of the concentration (the number of ultrafine bubbles/mL) of the ultrafine bubbles (preferably CO₂ ultrafine bubbles) in the melted water. More specific examples of such a concentration include 1 to 15 billion ultrafine bubbles/mL, 1 to 10 billion ultrafine bubbles/mL, 1 to 5 billion ultrafine bubbles/mL, 2 to 15 billion ultrafine bubbles/mL, 2 to 10 billion ultrafine bubbles/mL, 2 to 5 billion ultrafine bubbles/mL, 3 to 15 billion ultrafine bubbles/mL, 3 to 10 billion ultrafine bubbles/mL, 3 to 5 billion ultrafine bubbles/mL, 5 to 15 billion ultrafine bubbles/mL, 5 to 10 billion ultrafine bubbles/mL, 5 to 5 billion ultrafine bubbles/mL, 7 to 15 billion ultrafine bubbles/mL, 7 to 10 billion ultrafine bubbles/mL, 7 to 5 billion ultrafine bubbles/mL, 10 to 15 billion ultrafine bubbles/mL, 10 to 10 billion ultrafine bubbles/mL, and 10 to 5 billion ultrafine bubbles/mL. The CO₂-content rate of preferred consolidated CO₂ hydrate according to the present invention is preferably 7% by weight or more, more preferably 10% by weight or more, further preferably 13% by weight or more, more preferably 16% by weight or more, further preferably 18% by weight or more, from the viewpoint of obtaining a higher concentration of ultrafine bubbles and obtaining a higher muscle fatigue recovery promoting effect (preferably recovery promoting effect on induced muscle strength upon electrical stimulation). Examples of the upper limit value include, but are not particularly limited to, 30% by weight, 28% by weight, 26% by weight, and 24% by weight. More specific examples of the CO₂-content rate of preferred consolidated CO₂ hydrate according to the present invention include 7 to 30% by weight, 10 to 30% by weight, 13 to 30% by weight, 16 to 30% by weight, 18 to 30% by weight, 7 to 28% by weight, 10 to 28% by weight, 13 to 28% by weight, 16 to 28% by weight, 18 to 28% by weight, 7 to 26% by weight, 10 to 26% by weight, 13 to 26% by weight, 16 to 26% by weight, and 18 to 26% by weight.

Preferred examples of the method for producing the consolidated CO₂ hydrate according to the present invention can include, but are not particularly limited to, the following producing methods.

A conventional method such as a gas-liquid stirring technique of stirring raw material water while blowing CO₂ into the raw material water under conditions that meet CO₂ hydrate formation conditions, or a water spray technique of spraying raw material water into CO₂ under conditions that meet CO₂ hydrate formation conditions can be used. CO₂ hydrate formed by such a technique usually contains fine particles of the CO₂ hydrate in a slurry state mixed with unreacted water. The consolidated CO₂ hydrate can be produced by subjecting the slurry to dewatering treatment and compression treatment. As for the dewatering treatment and the compression treatment of the slurry containing CO₂ hydrate particles and water, for example, the dewatering treatment and the compression treatment may be separately performed in order in such a way that the dewatering treatment of the slurry is performed, and then, the compression treatment of the CO₂ hydrate particles is performed. Alternatively, the dewatering treatment and the compression treatment may be performed at the same time in such a way that the slurry is subjected to the compression treatment in a condition wherein water in the slurry is discharged. It is preferred to perform the dewatering treatment and the compression treatment at the same time from the viewpoint of obtaining a higher concentration of ultrafine bubbles and obtaining a higher muscle fatigue recovery promoting effect (preferably recovery promoting effect on induced muscle strength upon electrical stimulation). Among others, it is more preferred to perform the dewatering treatment and the compression treatment at the same time under CO₂ hydrate formation conditions. The compression treatment of the CO₂ hydrate particles or the compression treatment of the slurry can be performed using a commercially available compaction molding machine or the like. Examples of the pressure in the compression treatment can include 1 to 100 Mpa, 1 to 50 Mpa, 1 to 30 Mpa, 1 to 15 Mpa, 1 to 10 Mpa, and 2.5 to 10 Mpa. It has been reported that: sufficient dewatering treatment of the aforementioned slurry usually provides a CO₂ hydrate ratio of approximately 40%; the compression treatment of the CO₂ hydrate particles at 2.5 Mpa after sufficient dewatering treatment usually provides a CO₂ hydrate ratio of approximately 60%; and the compression treatment of the CO₂ hydrate particles at 10 Mpa after dewatering treatment usually provides a CO₂ hydrate ratio of approximately 70 to 90%.

The CO₂-rich ice (preferably CO₂ hydrate) in the muscle fatigue recovery-promoting agent of the present invention may be CO₂-rich ice (preferably CO₂ hydrate) consisting of CO₂ and ice (hereinafter, also referred to as “CO₂-rich ice (preferably CO₂ hydrate) containing no optional component”) or may be CO₂-rich ice (preferably CO₂ hydrate) further containing an optional component appropriate for the purpose of the muscle fatigue recovery-promoting agent. The muscle fatigue recovery-promoting agent of the present invention may be a muscle fatigue recovery-promoting agent consisting of the “CO₂-rich ice (preferably CO₂ hydrate) containing no optional component”, or the “CO₂-rich ice (preferably CO₂ hydrate) containing an optional component”, or may further contain an optional component in addition to such CO₂-rich ice (preferably CO₂ hydrate).

When the muscle fatigue recovery-promoting agent of the present invention contains CO₂-rich ice other than CO₂ hydrate, it is preferred that this muscle fatigue recovery-promoting agent of the present invention should be kept at a temperature and a pressure that do not melt ice during distribution or storage. Examples of such a temperature and a pressure include conditions of ordinary pressure (e.g., 1 atm) and 0° C. or lower. On the other hand, the CO₂ hydrate may be excellent in its preservability or stability depending on its producing method, etc. Thus, when the muscle fatigue recovery-promoting agent of the present invention contains CO₂ hydrate as the CO₂-rich ice, this muscle fatigue recovery-promoting agent of the present invention may be kept at ordinary temperature (5 to 35° C.) and ordinary pressure (e.g., 1 atm) during distribution or storage. It is preferred that the muscle fatigue recovery-promoting agent of the present invention should be kept “under a low-temperature condition” or “under a high-pressure condition”, or “under a low-temperature condition and under a high-pressure condition” during distribution, storage, etc., from the viewpoint of more stably maintaining the muscle fatigue recovery-promoting agent of the present invention for a longer period. Among them, it is preferred to keep the muscle fatigue recovery-promoting agent “under a low-temperature condition”, and it is more preferred to keep the muscle fatigue recovery-promoting agent at ordinary pressure (e.g., 1 atm) “under a low-temperature condition”, from the viewpoint of convenient keeping.

The upper limit temperature “under a low-temperature condition” described above is 10° C. or lower, preferably 5° C. or lower, more preferably 0° C. or lower, further preferably −5° C. or lower, more preferably −10° C. or lower, further preferably −15° C. or lower, more preferably −20° C., further preferably −25° C. Examples of the lower limit temperature “under a low-temperature condition” described above include −273° C. or higher, −80° C. or higher, −50° C. or higher, −40° C. or higher, and −30° C. or higher.

The lower limit pressure “under a high-pressure condition” described above is 1.036 atm or higher, preferably 1.135 atm or higher, more preferably 1.283 atm or higher, further preferably 1.480 atm or higher. Examples of the upper limit pressure “under a high-pressure condition” described above include 14.80 atm or lower, 11.84 atm or lower, 9.869 atm or lower, 7.895 atm or lower, and 4.935 atm or lower.

The muscle fatigue recovery-promoting agent of the present invention may be housed in a container. The container is not particularly limited by its shape or material. Examples thereof can include a plastic bottle container.

The muscle fatigue recovery-promoting agent of the present invention is not particularly limited by the degree of a concentration (the number of ultrafine bubbles/mL) of ultrafine bubbles that can be generated in water in ice water when the muscle fatigue recovery-promoting agent is added to water. In the case of adding 300 mg (based on ice having a CO₂-content rate of 3% by weight or more) of the muscle fatigue recovery-promoting agent of the present invention per mL of water, preferred examples of such a muscle fatigue recovery-promoting agent can include a muscle fatigue recovery-promoting agent that can generate preferably 5 million or more ultrafine bubbles/mL, more preferably 10 million or more ultrafine bubbles/mL, further preferably 20 million or more ultrafine bubbles/mL, more preferably 25 million or more ultrafine bubbles/mL, further preferably 30 million or more ultrafine bubbles/mL, more preferably 35 million or more ultrafine bubbles/mL, further preferably 50 million or more ultrafine bubbles/mL, more preferably 75 million or more ultrafine bubbles/mL, further preferably 1 hundred million or more ultrafine bubbles/mL, more preferably 150 million or more ultrafine bubbles/mL, further preferably 2 hundred million or more ultrafine bubbles/mL, more preferably 250 million or more ultrafine bubbles/mL in water in terms of the concentration (the number of ultrafine bubbles/mL) of the ultrafine bubbles (preferably CO₂ ultrafine bubbles) in water in the ice water.

Examples of the upper limit of the concentration of the ultrafine bubbles (preferably, CO₂ ultrafine bubbles) that can be generated by the muscle fatigue recovery-promoting agent of the present invention in water include, but are not particularly limited to, an ultrafine bubble concentration of 10 billion or less ultrafine bubbles/mL or 1 billion or less ultrafine bubbles/mL. More specific examples of the concentration of the ultrafine bubbles (preferably, CO₂ ultrafine bubbles) that can be generated by the muscle fatigue recovery-promoting agent of the present invention in water include 5 million to 10 billion ultrafine bubbles/mL, 5 million to 1 billion ultrafine bubbles/mL, 10 million to 10 billion ultrafine bubbles/mL, 10 million to 1 billion ultrafine bubbles/mL, 20 million to 10 billion ultrafine bubbles/mL, 20 million to 1 billion ultrafine bubbles/mL, 25 million to 10 billion ultrafine bubbles/mL, 25 million to 1 billion ultrafine bubbles/mL, 30 million to 10 billion ultrafine bubbles/mL, 30 million to 1 billion ultrafine bubbles/mL, 35 million to 10 billion ultrafine bubbles/mL, 35 million to 1 billion ultrafine bubbles/mL, 50 million to 10 billion ultrafine bubbles/mL, 50 million to 1 billion ultrafine bubbles/mL, 75 million to 10 billion ultrafine bubbles/mL, 75 million to 1 billion ultrafine bubbles/mL, 1 hundred million to 10 billion ultrafine bubbles/mL, 1 hundred million to 1 billion ultrafine bubbles/mL, 150 million to 10 billion ultrafine bubbles/mL, 150 million to 1 billion ultrafine bubbles/mL, 2 hundred million to 10 billion ultrafine bubbles/mL, 2 hundred million to 1 billion ultrafine bubbles/mL, 250 million to 10 billion ultrafine bubbles/mL, and 250 million to 1 billion ultrafine bubbles/mL.

In the case of adding the muscle fatigue recovery-promoting agent of the present invention to water, the measurement value of the ultrafine bubble concentration in water in the ice water is preferably a measurement value according to the aforementioned measurement method R, more preferably a measurement value according to the following measurement method R2.

### (Measurement Method R2)

The muscle fatigue recovery-promoting agent of −80 to 0° C. is added at 300 mg/mL (based on ice having a CO₂-content rate of 3% by weight or more) to water of 25° C. to prepare ice water of 0 to 2° C. containing ice having a CO₂-content rate of 3% by weight or more. Then, the concentration (the number of ultrafine bubbles/mL) of the ultrafine bubbles in water in the ice water is measured by a laser diffraction/scattering method (preferably quantitative laser diffraction/scattering method) or a nanotracking method.

### (Optional Component)

The muscle fatigue recovery-promoting agent of the present invention contains CO₂-rich ice as an essential component and may further contain an optional component without interfering with the effect of the present invention (muscle fatigue recovery promoting effect, preferably recovery promoting effect on induced muscle strength upon electrical stimulation). Examples of such an optional component include a component having drug efficacy and an additive. Examples of such a component having drug efficacy include other muscle fatigue recovery-promoting agents, an analgesic, and an antiphlogistic. Examples of the additive described above include a fragrance, a colorant, a thickener, and a pH adjuster.

### (Subject)

Preferred examples of the type of the animal that is subject to the muscle fatigue recovery-promoting agent or the muscle fatigue recovery-promoting liquid of the present invention include, but are not particularly limited to, an animal belonging to any class selected from the group consisting of a mammal, bird, a reptile, an amphibia, and fish, more preferably an animal belonging to a mammal or bird, further preferably an animal belonging to a mammal, more preferably a human, a dog, a cat, a horse, a pony, a donkey, cattle, a pig, sheep, a goat, a rabbit, a monkey, a mouse, a rat, a hamster, a guinea pig, and a ferret, further preferably a human and a horse, particularly preferably a human. The horse is preferably a thoroughbred which is a racing horse. This is because the racing horse runs full pelt in races and therefore have very severe muscle fatigue after races.

Examples of the animal that is subject to the muscle fatigue recovery-promoting agent or the muscle fatigue recovery-promoting liquid of the present invention include an animal having fatigue of a portion or the whole of muscles. More specific examples of the purpose of the muscle fatigue recovery-promoting agent or the muscle fatigue recovery-promoting liquid of the present invention include: an agent for muscle fatigue recovery promotion in the body before exercise, during exercise, and/or after exercise; and an agent for recovery promotion of chronic muscle fatigue in the body.

### (Location of Application)

Examples of the location of application of the muscle fatigue recovery-promoting agent or the muscle fatigue recovery-promoting liquid of the present invention include the systemic or local skin of an animal. In the present specification, the term “systemic” means the whole body within a range that can secure breathing of an animal when the muscle fatigue recovery-promoting agent or the muscle fatigue recovery-promoting liquid of the present invention is applied to the animal. For a mammal, the term usually means that the skin except for the head, i.e., the skin below the neck, is immersed in the muscle fatigue recovery-promoting agent or the muscle fatigue recovery-promoting liquid of the present invention. In the present specification, the term “local” means, but is not particularly limited to, any body part, for example, the head, the face, the neck, the shoulder, the arm, the hand, the chest, the abdomen, the hip, the leg, or the foot. The muscle fatigue recovery-promoting agent or the muscle fatigue recovery-promoting liquid of the present invention may be applied to a plurality of body parts at the same time. In the present specification, the “skin” is not particularly limited as long as the skin is the skin of an animal. The skin also includes the mucosa of an animal for the sake of convenience. Examples of the mucosa include the lips and oral mucosa.

### (Method of Using Muscle Fatigue Recovery-Promoting Agent)

Preferred examples of the method for using the muscle fatigue recovery-promoting agent of the present invention include a method of applying the muscle fatigue recovery-promoting agent of the present invention to the skin (i.e., contacting the muscle fatigue recovery-promoting agent of the present invention with the skin), and a method of applying the muscle fatigue recovery-promoting liquid of the present invention to the skin (i.e., contacting the muscle fatigue recovery-promoting liquid of the present invention with the skin), the muscle fatigue recovery-promoting liquid being produced by contacting the muscle fatigue recovery-promoting agent with a liquid or melting the muscle fatigue recovery-promoting agent as it is, and more specifically include a method of directly applying the muscle fatigue recovery-promoting agent of the present invention to the skin (i.e., directly contacting the muscle fatigue recovery-promoting agent of the present invention with the skin) (hereinafter, also referred to as “method 1”), a method of applying the muscle fatigue recovery-promoting agent of the present invention to the skin via a fibrous material (i.e., contacting the muscle fatigue recovery-promoting agent of the present invention with a fibrous material and contacting the fibrous material with the skin) (hereinafter, also referred to as “method 2”), and a method of applying the muscle fatigue recovery-promoting liquid of the present invention to the skin (i.e., contacting the muscle fatigue recovery-promoting liquid of the present invention with the skin) (hereinafter, also referred to as “method 3”). The muscle fatigue recovery-promoting agent of the present invention is preferably used to prepare before use a muscle fatigue recovery-promoting liquid for application to the skin, as mentioned above.

The above method 1 is not particularly limited as long as the method involves directly applying the muscle fatigue recovery-promoting agent of the present invention to the skin (i.e., directly contacting the muscle fatigue recovery-promoting agent of the present invention with the skin). Examples thereof include a method of placing the muscle fatigue recovery-promoting agent of the present invention in a container such as a bucket, and placing the skin at the desired site in the muscle fatigue recovery-promoting agent of the present invention in the container, and a method of placing the muscle fatigue recovery-promoting agent of the present invention in a shape-changeable container, and fixing the container near the skin such that the recovery promoting agent is contacted with the skin at the desired site. When the muscle fatigue recovery-promoting agent of the present invention is contacted with the skin, for example, a portion of the CO₂-rich ice is melted to yield the muscle fatigue recovery-promoting liquid of the present invention. As a result, not only the muscle fatigue recovery-promoting agent of the present invention but the muscle fatigue recovery-promoting liquid of the present invention is directly contacted with the skin.

The above method 2 is not particularly limited as long as the method involves applying the muscle fatigue recovery-promoting agent of the present invention to the skin via a fibrous material (i.e., contacting the muscle fatigue recovery-promoting agent of the present invention with a fibrous material and contacting the fibrous material with the skin).

The fibrous material in the above method 2 is not particularly limited by its material, its shape, whether to be a woven fabric, to be a nonwoven fabric, or to be a sponge, etc. as long as melted water of the CO₂-rich ice (preferably CO₂ hydrate), when contacted with one surface of the fibrous material, is capable of penetrating the fibrous material into the other surface of the fibrous material.

Examples of the material of the fibrous material described above can include natural fibers such as cotton, wool, cupra, silk, kapok, flax, hemp, jute, ramie, kenaf, abaca cloth, and palm, synthetic fibers such as nylon, polypropylene, polyethylene, polyamide, polyester, polyacryl, and polyurethane, and blend fibers thereof. Among them, cotton is preferred.

When the muscle fatigue recovery-promoting agent of the present invention is contacted with the skin via the fibrous material, a portion of the CO₂-rich ice is melted to yield the muscle fatigue recovery-promoting liquid of the present invention. As a result, the recovery promoting liquid penetrates the fibrous material and is thereby directly contacted with the skin.

Examples of the shape of the fibrous material described above include a sheet-like shape and a sac-like shape. A sac-like shape is preferred from the viewpoint of easy use. The muscle fatigue recovery-promoting agent of the present invention is placed in, for example, a long sac-like fibrous material, and the long sac-like fibrous material is wrapped around a body part such as the leg so that the “muscle fatigue recovery-promoting agent of the present invention” or “melted water of the CO₂-rich ice (preferably CO₂ hydrate) contained in the agent” can be stably contacted with the skin of the body part.

The above method 3 is not particularly limited as long as the method involves applying the muscle fatigue recovery-promoting liquid of the present invention to the skin (i.e., contacting the muscle fatigue recovery-promoting liquid of the present invention with the skin). Examples thereof include a method of placing the muscle fatigue recovery-promoting liquid of the present invention in a container such as a bucket, and placing the skin at the desired site in the muscle fatigue recovery-promoting liquid of the present invention in the container, and a method of placing the muscle fatigue recovery-promoting liquid of the present invention in a shape-changeable container, and fixing the container near the skin such that the recovery promoting liquid is contacted with the skin at the desired site.

The amount of the muscle fatigue recovery-promoting agent of the present invention used can be appropriately set according to the area of the skin to which the muscle fatigue recovery-promoting agent or the muscle fatigue recovery-promoting liquid of the present invention is applied, the severity of muscle fatigue, the method for using the muscle fatigue recovery-promoting agent, etc. For example, in the above method 1, 0.3 to 30 g, preferably 1 to 25 g, more preferably 2 to 10 g (based on CO₂-rich ice (preferably CO₂ hydrate)) of the muscle fatigue recovery-promoting agent of the present invention is used per 25 cm² of the skin. In the above method 2, 0.3 to 30 g, preferably 1 to 25 g, more preferably 2 to 10 g (based on CO₂-rich ice (preferably CO₂ hydrate)) of the muscle fatigue recovery-promoting agent of the present invention is used per 25 cm² of the fibrous material. In the above method 3, examples of the amount of the CO₂-rich ice used (preferably the amount of the CO₂-rich ice added) (mg/mL) in preparing the muscle fatigue recovery-promoting liquid of the present invention include an amount described in a section about the producing method of the present invention mentioned later.

### (Temperature in Use)

The temperature of the muscle fatigue recovery-promoting liquid of the present invention in applying the muscle fatigue recovery-promoting liquid to the skin can be appropriately set. Examples thereof include 0 to 20° C., 0 to 15° C., 0 to 10° C., 0 to 8° C., 0 to 6° C., 0 to 4° C., 0 to 3° C., 0 to 2° C., 2 to 20° C., 2 to 15° C., 2 to 10° C., 2 to 8° C., 2 to 6° C., 2 to 4° C., 4 to 20° C., 4 to 15° C., 4 to 10° C., 4 to 8° C., and 4 to 6° C. Examples of the method for adjusting the temperature of the muscle fatigue recovery-promoting liquid of the present invention include, but are not particularly limited to, a method of adjusting the temperature of a liquid to be contacted with the muscle fatigue recovery-promoting agent, and a method of adjusting the temperature of the muscle fatigue recovery-promoting liquid using a cooling apparatus or a heating apparatus. A commercially available product can be used as the cooling apparatus or the heating apparatus.

The CO₂-rich ice (preferably CO₂ hydrate) in the muscle fatigue recovery-promoting agent of the present invention is usually a solid. When the muscle fatigue recovery-promoting liquid is prepared by contacting CO₂-rich ice with a liquid, a portion of the CO₂-rich ice or the CO₂-rich ice, when melted, draws a great deal of heat from the liquid. Therefore, the temperature of the liquid is relatively drastically decreased. Thus, for preparing the muscle fatigue recovery-promoting liquid by contacting the muscle fatigue recovery-promoting agent of the present invention with a liquid, it is preferred to use a liquid having a higher temperature than the desired temperature of the muscle fatigue recovery-promoting liquid. For example, it is preferred to use a liquid having a temperature higher by 2° C. or more, 4° C. or more or 6° C. or more than the desired temperature of the muscle fatigue recovery-promoting liquid to be prepared. It is also preferred that the muscle fatigue recovery-promoting liquid of the present invention should be prepared before use for application to the skin from the viewpoint of obtaining a higher muscle fatigue recovery promoting effect (preferably recovery promoting effect on induced muscle strength upon electrical stimulation). In the present specification, the phrase “prepare before use” includes the preparation of the muscle fatigue recovery-promoting liquid of the present invention, for example, within 1 hour before, preferably within 40 minutes before, more preferably within 30 minutes before, further preferably within 20 minutes before, more preferably within 10 minutes before, further preferably within 5 minutes before the start of application of the muscle fatigue recovery-promoting liquid to the skin (the start of contact of the muscle fatigue recovery-promoting liquid with the skin).

The surface temperature of the muscle fatigue recovery-promoting agent of the present invention in applying the muscle fatigue recovery-promoting agent to the skin or the fibrous material can be appropriately set and may be lower than −20 to 0° C. Examples thereof include 0 to 3° C.

### (Application Time)

The application time of the muscle fatigue recovery-promoting agent or the muscle fatigue recovery-promoting liquid of the present invention is not particularly limited and can be appropriately set as long as the effect of the present invention (muscle fatigue recovery promoting effect, preferably recovery promoting effect on induced muscle strength upon electrical stimulation) is obtained. Examples thereof include 3 to 30 minutes, 5 to 25 minutes, 5 to 20 minutes, 5 to 15 minutes, 10 to 20 minutes, and 10 to 15 minutes for which the muscle fatigue recovery-promoting agent or the muscle fatigue recovery-promoting liquid of the present invention is contacted with the skin at the location of application.

### (Application Frequency)

The application frequency of the muscle fatigue recovery-promoting agent or the muscle fatigue recovery-promoting liquid of the present invention is not particularly limited and can be appropriately determined on the basis of improvement in symptom, etc. Examples thereof include a frequency on the order of once to three times per day to 3 days.

### (Muscle Fatigue Recovery Promoting Effect)

In the present specification, the CO₂-rich ice, the muscle fatigue recovery-promoting agent of the present invention, or the muscle fatigue recovery-promoting liquid of the present invention (hereinafter, also collectively referred to as “CO₂-rich ice, etc.”) “has a muscle fatigue recovery promoting effect”. This means that the CO₂-rich ice, etc. applied to the skin at the site of a fatigued muscle promotes the recovery of fatigue of the muscle when compared with no treatment of a fatigued muscle or usual ice applied to a fatigued muscle. Preferred examples of the index for the recovery of fatigue of such a muscle include, but are not particularly limited to, the recovery of induced muscle strength upon electrical stimulation to the muscle.

The phrase “have a muscle fatigue recovery promoting effect” as to the CO₂-rich ice, etc. includes the suppression of reduction in induced muscle strength caused by fatigue when the CO₂-rich ice, etc. is applied to a fatigued muscle (e.g., when the CO₂-rich ice, etc. is applied thereto for 15 to 25 minutes or for 20 minutes) as compared with when no treatment is performed for a fatigued muscle (e.g., when a fatigued muscle is left for 15 to 25 minutes or for 20 minutes) or when usual ice is applied to a fatigued muscle (e.g., when usual ice is applied thereto for 15 to 25 minutes or for 20 minutes). The suppression of reduction in induced muscle strength caused by fatigue includes suppression to 8 or less, preferably 5 or less, more preferably 3 or less, further preferably 2 or less, in terms of the degree (relative value to a reference value of 10) of reduction in induced muscle strength caused by fatigue, by the post-fatigue application of the CO₂-rich ice, etc. when the degree of reduction in induced muscle strength caused by fatigue without treatment of a fatigued muscle is defined as 10 (reference value), and most preferably includes the recovery of induced muscle strength to pre-fatigue induced muscle strength by the post-fatigue application of the CO₂-rich ice, etc.

### 2. <Method for Producing Muscle Fatigue Recovery-Promoting Liquid of Present Invention>

The method for producing the muscle fatigue recovery-promoting liquid of the present invention (the producing of the present invention) is not particularly limited as long as the method comprises the step of contacting “ice (preferably CO₂ hydrate) having a CO₂-content rate of 3% by weight or more” (or the “muscle fatigue recovery-promoting agent of the present invention”) with a liquid or melting the ice or the muscle fatigue recovery-promoting agent as it is. A muscle fatigue recovery-promoting liquid containing CO₂ bubbles (preferably ultrafine bubbles) can be produced by contacting the CO₂-rich ice (preferably CO₂ hydrate) with a liquid or melting the CO₂-rich ice as it is.

### (Liquid)

The “liquid” according to the present invention is not particularly limited as long as the liquid enables the CO₂-rich ice (preferably CO₂ hydrate) to generate CO₂ bubbles (preferably ultrafine bubbles) when the CO₂-rich ice (preferably CO₂ hydrate) is contained in the liquid, and may be contacted with the skin of an animal. Examples thereof include (i) a “hydrophilic solvent”, (ii) a “hydrophobic solvent”, (iii) a “hydrophilic solvent/hydrophobic solvent mixed solvent”, and a “liquid containing an arbitrary solute in any of the solvents (i) to (iii)”. A temperature condition and a pressure condition under which the “liquid” according to the present invention is in a liquid state vary depending on the type of the solvent, the purpose of the liquid, use conditions of the liquid, etc., and therefore cannot be generalized. Preferred examples of such a liquid include a liquid that is in a liquid state under conditions of 20° C. and 1 atm.

The “hydrophilic solvent” used in the present invention has a solubility parameter (SP value) of preferably 20 or more, further preferably 29.9 or more. Specifically, it is preferred to use one or more solvents selected from the group consisting of water (47.9), a polyhydric alcohol, and a lower alcohol. Examples of the polyhydric alcohol include a dihydric alcohol such as ethylene glycol (29.9), diethylene glycol (24.8), triethylene glycol (21.9), tetraethylene glycol (20.3), and propylene glycol (25.8), a trihydric alcohol such as glycerin (33.8), diglycerin, triglycerin, polyglycerin and trimethylolpropane, a tetrahydric or higher alcohol such as diglycerin, triglycerin, polyglycerin, pentaerythritol, and sorbitol, hexitol such as sorbitol, aldose such as glucose, a compound having a sugar skeleton such as sucrose, and others. Examples of the lower alcohol include isopropanol (23.5), butyl alcohol (23.3), and ethyl alcohol (26.9). Two or more of these hydrophilic solvents may be used in combination. The δ value of the solubility parameter is shown within the parentheses. A preferred hydrophilic solvent according to the present invention preferably contains water and is more preferably water.

The “hydrophobic solvent” used in the present invention is preferably an organic solvent having a solubility parameter (SP value) of less than 20.0 and, specifically, is preferably a hydrocarbon-based solvent or a silicone-based solvent, or a mixture thereof. Examples of the hydrocarbon-based solvent can include an aliphatic hydrocarbon such as hexane (14.9), heptane (14.3), dodecane (16.2), cyclohexane (16.8), methylcyclohexane (16.1), octane (16.0), and hydrogenated triisobutylene, an aromatic hydrocarbon such as benzene (18.8), toluene (18.2), ethylbenzene (18.0), and xylene (18.0), and a halogenated hydrocarbon such as chloroform (19.3), 1,2-dichloromethane (19.9), and trichloroethylene (19.1). Examples of the silicone-based solvent include octamethylcyclotetrasiloxane, decamethylcyclopentasiloxane, hexamethyldisiloxane, and octamethyltrisiloxane. Among them, hexane (14.9) and cyclohexane (16.8) are particularly preferred. Two or more of these hydrophobic solvents may be used in combination.

The “solute” in the “liquid containing an arbitrary solute in any of the solvents (i) to (iii)” described above is not particularly limited as long as the solute enables the CO₂-rich ice (preferably CO₂ hydrate) to generate CO₂ bubbles (preferably ultrafine bubbles) when the CO₂-rich ice (preferably CO₂ hydrate) is contained in the liquid. Examples thereof include carbon dioxide and common salt. Specific examples of the “liquid containing an arbitrary solute in any of the solvents (i) to (iii)” include melted water of the CO₂-rich ice and physiological saline, preferably melted water of the CO₂-rich ice, more preferably melted water of CO₂ hydrate. The melted water of the CO₂-rich ice such as CO₂ hydrate contains carbon dioxide as the solute.

In the present specification, the “muscle fatigue recovery-promoting liquid” is not necessarily required to be in a liquid state as a whole and also includes a mixture of solid CO₂-rich ice (preferably CO₂ hydrate) and a liquid.

In the present specification, the method for “contacting ice (preferably CO₂ hydrate) having a CO₂-content rate of 3% by weight or more with a liquid” is not particularly limited as long as the method attains the contact between CO₂-rich ice (preferably CO₂ hydrate) and the liquid. Preferred examples thereof include a method of allowing the CO₂-rich ice (preferably CO₂ hydrate) to be contained in the liquid. Among others, a method of adding or injecting the CO₂-rich ice (preferably CO₂ hydrate) to the liquid and a method of adding or injecting the liquid to the CO₂-rich ice (preferably CO₂ hydrate) are more preferred, and a method of adding or injecting the CO₂-rich ice (preferably CO₂ hydrate) to the liquid is further preferred.

In the producing method of the present invention, the amount of the CO₂-rich ice used (preferably the amount of the CO₂-rich ice added) (mg/mL) in contacting the CO₂-rich ice with the liquid can be appropriately set by those skilled in the art according to whether or not the CO₂-rich ice is CO₂ hydrate, whether or not to be consolidated CO₂ hydrate, the CO₂-content rate of the CO₂-rich ice, or a necessary degree of a concentration of CO₂ bubbles (preferably ultrafine bubbles). Examples of the lower limit of the amount of the CO₂-rich ice used (preferably the amount of the CO₂-rich ice added) (mg/mL) include 10 mg/mL or more. The lower limit is preferably 20 mg/mL or more, more preferably 50 mg/mL or more, further preferably 100 mg/mL or more, more preferably 150 mg/mL or more, further preferably 200 mg/mL or more, from the viewpoint of obtaining a higher concentration of CO₂ bubbles (preferably ultrafine bubbles). Examples of the upper limit of the amount of the CO₂-rich ice used (preferably the amount of the CO₂-rich ice added) (mg/mL) include, but are not particularly limited to, 5000 mg/mL or less, 3000 mg/mL or less, 2000 mg/mL or less, 1000 mg/mL or less, and 500 mg/mL or less. More specific examples of the amount of the CO₂-rich ice used (preferably the amount of the CO₂-rich ice added) (mg/mL) include 20 to 5000 mg/mL, 20 to 3000 mg/mL, 20 to 2000 mg/mL, 50 to 2000 mg/mL, 50 to 1000 mg/mL, 100 to 500 mg/mL, and 150 to 500 mg/mL. The amount of the CO₂-rich ice used (mg/mL) means the weight (mg) of the CO₂-rich ice to be used (preferably to be added) per mL of the liquid.

The temperature of the liquid in contacting the CO₂-rich ice with the liquid is not particularly limited as long as CO₂ bubbles (preferably ultrafine bubbles) are generated. Examples thereof include 0 to 50° C., 0 to 35° C., 0 to 25° C., 0 to 20° C., 0 to 15° C., 0 to 10° C., 0 to 7° C., 0 to 5° C., 3 to 50° C., 3 to 35° C., 3 to 25° C., 3 to 15° C., 3 to 10° C., 3 to 7° C., 3 to 5° C., 6 to 50° C., 6 to 35° C., 6 to 25° C., 6 to 20° C., 6 to 15° C., 6 to 10° C., 6 to 7° C., 10 to 50° C., 10 to 35° C., 10 to 25° C., and 10 to 15° C. Those skilled in the art can appropriately set the temperature according to the temperature of the muscle fatigue recovery-promoting liquid they desire. The CO₂-rich ice (preferably CO₂ hydrate) in the muscle fatigue recovery-promoting agent of the present invention is usually a solid. When the muscle fatigue recovery-promoting liquid is prepared by contacting CO₂-rich ice with a liquid, a portion of the CO₂-rich ice or the CO₂-rich ice, when melted, draws a great deal of heat from the liquid. Therefore, the temperature of the liquid is relatively drastically decreased. Thus, for preparing the muscle fatigue recovery-promoting liquid by contacting the muscle fatigue recovery-promoting agent of the present invention with a liquid, it is preferred to use a liquid having a higher temperature than the desired temperature of the muscle fatigue recovery-promoting liquid. For example, it is preferred to use a liquid having a temperature higher by 2° C. or more, 4° C. or more or 6° C. or more, preferably a liquid having a temperature higher by 2 to 10° C., 4 to 10° C. or 6 to 10° C., than the desired temperature of the muscle fatigue recovery-promoting liquid to be prepared.

In the present specification, the method for “melting ice (preferably CO₂ hydrate) having a CO₂-content rate of 3% by weight or more as it is” is not particularly limited as long as the method of exposing the CO₂-rich ice (preferably CO₂ hydrate) under a temperature condition that melts the CO₂-rich ice (preferably CO₂ hydrate). Examples thereof include a method of placing the CO₂-rich ice (preferably CO₂ hydrate) in a container and leaving the container under a condition of 1 to 30° C.

In the producing method of the present invention, examples of the amount of the CO₂-rich ice used in melting the CO₂-rich ice as it is can include the same weight as a necessary weight of the muscle fatigue recovery-promoting liquid.

### 3. <Muscle Fatigue Recovery-Promoting Liquid of Present Invention>

The muscle fatigue recovery-promoting liquid of the present invention is a muscle fatigue recovery-promoting liquid for application to the skin. The muscle fatigue recovery-promoting liquid of the present invention is not particularly limited as long as the liquid comprises 200 ppm or more of carbonic acid and contains 5 million or more ultrafine bubbles/mL. A muscle fatigue recovery-promoting liquid produced by the producing method of the present invention is preferred.

In the present specification, the “muscle fatigue recovery-promoting liquid” is not necessarily required to be in a liquid state as a whole and also includes a mixture of solid CO₂-rich ice (preferably CO₂ hydrate) and a liquid, as mentioned above.

The muscle fatigue recovery-promoting liquid of the present invention is not particularly limited as long as the liquid comprises 200 ppm or more of carbonic acid. It is preferred that the muscle fatigue recovery-promoting liquid of the present invention should comprise preferably 500 ppm (0.05% by weight) or more, more preferably 750 ppm (0.075% by weight) or more, further preferably 900 ppm (0.09% by weight) or more, more preferably 1000 ppm (0.1% by weight) or more, of carbonic acid. Examples of the upper limit of the carbonic acid concentration include, but are not particularly limited to, 5000 ppm (0.5% by weight) or less, 4000 ppm (0.4% by weight) or less, 3000 ppm (0.3% by weight) or less, 2000 ppm (0.2% by weight) or less, and 1500 ppm (0.15% by weight) or less. More specific examples of the carbonic acid concentration in the muscle fatigue recovery-promoting liquid of the present invention include 500 to 5000 ppm, 750 to 5000 ppm, 900 to 5000 ppm, 1000 to 5000 ppm, 500 to 4000 ppm, 750 to 4000 ppm, 900 to 4000 ppm, 1000 to 4000 ppm, 500 to 3000 ppm, 750 to 3000 ppm, 900 to 3000 ppm, 1000 to 3000 ppm, 500 to 2000 ppm, 750 to 2000 ppm, 900 to 2000 ppm, 1000 to 2000 ppm, 500 to 1500 ppm, 750 to 1500 ppm, 900 to 1500 ppm, and 1000 to 1500 ppm.

The carbonic acid concentration in the muscle fatigue recovery-promoting liquid of the present invention means a concentration measured at a liquid temperature of 0 to 2° C. under ordinary pressure.

The value of the concentration of the ultrafine bubbles (preferably CO₂ ultrafine bubbles) in the muscle fatigue recovery-promoting liquid of the present invention is not particularly limited as long as the value is 5 million or more ultrafine bubbles/mL. The value is preferably 10 million or more ultrafine bubbles/mL, more preferably 20 million or more ultrafine bubbles/mL, further preferably 25 million or more ultrafine bubbles/mL, more preferably 30 million or more ultrafine bubbles/mL, further preferably 35 million or more ultrafine bubbles/mL, more preferably 50 million or more ultrafine bubbles/mL, further preferably 75 million or more ultrafine bubbles/mL, more preferably 1 hundred million or more ultrafine bubbles/mL, further preferably 150 million or more ultrafine bubbles/mL, more preferably 2 hundred million or more ultrafine bubbles/mL, further preferably 250 million or more ultrafine bubbles/mL. Examples of the upper limit of the concentration of the ultrafine bubbles (preferably CO₂ ultrafine bubbles) in the muscle fatigue recovery-promoting liquid of the present invention include, but are not particularly limited to, 10 billion or less ultrafine bubbles/mL and 1 billion or less ultrafine bubbles/mL. More specific examples of the concentration of the ultrafine bubbles (preferably CO₂ ultrafine bubbles) in the muscle fatigue recovery-promoting liquid of the present invention include 5 million to 10 billion ultrafine bubbles/mL, 5 million to 1 billion ultrafine bubbles/mL, 10 million to 10 billion ultrafine bubbles/mL, 10 million to 1 billion ultrafine bubbles/mL, 20 million to 10 billion ultrafine bubbles/mL, 20 million to 1 billion ultrafine bubbles/mL, 25 million to 10 billion ultrafine bubbles/mL, 25 million to 1 billion ultrafine bubbles/mL, 30 million to 10 billion ultrafine bubbles/mL, 30 million to 1 billion ultrafine bubbles/mL, 35 million to 10 billion ultrafine bubbles/mL, 35 million to 1 billion ultrafine bubbles/mL, 50 million to 10 billion ultrafine bubbles/mL, 50 million to 1 billion ultrafine bubbles/mL, 75 million to 10 billion ultrafine bubbles/mL, 75 million to 1 billion ultrafine bubbles/mL, 1 hundred million to 10 billion ultrafine bubbles/mL, 1 hundred million to 1 billion ultrafine bubbles/mL, 150 million to 10 billion ultrafine bubbles/mL, 150 million to 1 billion ultrafine bubbles/mL, 2 hundred million to 10 billion ultrafine bubbles/mL, 2 hundred million to 1 billion ultrafine bubbles/mL, 250 million to 10 billion ultrafine bubbles/mL, and 250 million to 1 billion ultrafine bubbles/mL.

The value of the concentration of the ultrafine bubbles (preferably CO₂ ultrafine bubbles) in the muscle fatigue recovery-promoting liquid of the present invention may be a measurement value of any measurement method that can measure the concentration of the ultrafine bubbles, and is preferably a measurement value according to the aforementioned measurement method R, more preferably a measurement value according to the aforementioned measurement method R1 or R2.

The temperature of the muscle fatigue recovery-promoting liquid of the present invention can be appropriately set according to the state of a muscle at the location of application, etc. Examples thereof include 0 to 20° C., 0 to 15° C., 0 to 10° C., 0 to 8° C., 0 to 6° C., 0 to 4° C., 0 to 3° C., 0 to 2° C., 2 to 20° C., 2 to 15° C., 2 to 10° C., 2 to 8° C., 2 to 6° C., 2 to 4° C., 4 to 20° C., 4 to 15° C., 4 to 10° C., 4 to 8° C., and 4 to 6° C.

The method for producing the muscle fatigue recovery-promoting liquid of the present invention is as described in the above section “2.”.

The muscle fatigue recovery-promoting liquid of the present invention may be housed in a container. The container is not particularly limited by its shape or material. Examples thereof can include a plastic bottle container.

### 4. <Muscle Fatigue Recovery Promotion Method of Present Invention>

The muscle fatigue recovery promotion method of the present invention is a method for promoting the recovery of muscle fatigue in an animal. The muscle fatigue recovery promotion method of the present invention is not particularly limited as long as the method comprises the step of applying CO₂-rich ice (preferably CO₂ hydrate) or the muscle fatigue recovery-promoting agent or the muscle fatigue recovery-promoting liquid of the present invention to the systemic or local skin of an animal (e.g., a nonhuman animal).

Preferred examples of the method for applying (e.g., contacting) CO₂-rich ice (preferably CO₂ hydrate) or the muscle fatigue recovery-promoting agent or the muscle fatigue recovery-promoting liquid of the present invention to the systemic or local skin of an animal include the aforementioned methods 1 to 3, i.e., a method of directly applying the muscle fatigue recovery-promoting agent of the present invention to the skin (i.e., directly contacting the muscle fatigue recovery-promoting agent of the present invention with the skin) (“method 1”), a method of applying the muscle fatigue recovery-promoting agent of the present invention to the skin via a fibrous material (i.e., contacting the muscle fatigue recovery-promoting agent of the present invention with a fibrous material and contacting the fibrous material with the skin) (“method 2”), and a method of applying the muscle fatigue recovery-promoting liquid of the present invention to the skin (i.e., contacting the muscle fatigue recovery-promoting liquid of the present invention with the skin) (“method 3”).

Hereinafter, the present invention will be described in detail with reference to Examples. However, the present invention is in no way limited to these Examples.

### EXAMPLE 1

### Test 1. [Preparation of CO₂ Hydrate]

CO₂ gas was blown at 3 MPa into 4 L of water, and CO₂ hydrate formation reaction was allowed to proceed at 1° C. with stirring to obtain “CO₂ hydrate slurry” containing CO₂ hydrate particles suspended in water. The slurry was poured into a cylinder-type compaction molding machine and compressed at a pressing pressure of 1 MPa at maximum for 3 minutes to remove water from the CO₂ hydrate slurry. Then, the CO₂ hydrate particles were pressed at a pressure of 10 MPa and then cooled to −20° C. A cylindrical mass of consolidated CO₂ hydrate was recovered from the compaction molding machine, followed by the crushing of the cylindrical mass. Consolidated CO₂ hydrate in a polyhedral shape having a maximum length of 3 mm or larger and 60 mm or smaller (hereinafter, simply referred to as “CO₂ hydrate” in this Example) was selectively recovered and used in subsequent experiments. This CO₂ hydrate had a CO₂-content rate of 20 to 25% and a CO₂ hydrate ratio of approximately 72 to 89%. The concentration (the number of ultrafine bubbles/mL) of ultrafine bubbles in melted water of the CO₂ hydrate was measured using “NanoSight NS300” manufactured by Malvern Panalytical Ltd. and was consequently approximately 13 hundred million ultrafine bubbles/mL. The carbonic acid concentration of the melted water of the CO₂ hydrate was 2000 ppm or more of carbonic acid contained.

Test 2. [Confirmation of Muscle Fatigue Recovery Promoting Effect Brought about by Contacting CO₂ Hydrate or Melted Water Thereof with Skin]

Voluntary ankle joint plantar flexion exercise was repetitively carried out targeting a total of 36 healthy adult males to cause a given level of fatigue in triceps surae muscle (medial head of gastrocnemius muscle, lateral head of gastrocnemius muscle, soleus muscle). Then, the CO₂ hydrate was contacted with the triceps surae muscle and evaluated for a muscle fatigue recovery-promoting effect brought about thereby. The specific method used therefor was a method mentioned later with reference to the method described in Akagi et al., Frontiers in Physiology (2017) Volume 8 Article 708. The index for muscle fatigue used was “induced torque (induced muscle strength) upon electrical stimulation” known as an index for evaluating peripheral fatigue.

Three groups each involving 12 test subjects (a total of 36 individuals) were provided. These three groups were set to a CO₂ hydrate group, an ice group, and a noncontact group, respectively. Forty rounds×2 sets of “exertion of ankle joint plantar flexor strength at full power for 3 seconds—rest for 3 seconds” (rest between the sets: 1 min) were carried out as a fatigue task for the test subjects in each group. Then, gauze of 5 cm square moistened with CO₂ hydrate melted water was placed on the skin of each muscle (medial head of gastrocnemius muscle, lateral head of gastrocnemius muscle, soleus muscle) at 3 sites of the triceps surae muscle of each test subject in the CO₂ hydrate group, and 5 g of CO₂ hydrate per site of the muscle (a total of 15 g for the 3 sites) was placed on the gauze so that the CO₂ hydrate (and CO₂ hydrate melted water obtained by melting thereof) was contacted for 20 minutes with the skin at each site of the triceps surae muscle. As for the ice group, gauze of 5 cm square moistened with water was placed on the skin of each muscle (medial head of gastrocnemius muscle, lateral head of gastrocnemius muscle, soleus muscle) at 3 sites of the triceps surae muscle of each test subject, and 5 g of ice per site of the muscle (a total of 15 g for the 3 sites) was placed on the gauze so that the ice (and CO₂ hydrate melted water obtained by melting thereof) was contacted for 20 minutes with the skin at each site of the triceps surae muscle. As for the noncontact group, the aforementioned fatigue task was carried out, and then, the test subjects were left for 20 minutes without contact with CO₂ hydrate and ice.

For the CO₂ hydrate group and the ice group, pre-fatigue (i.e., immediately before carrying out the fatigue task) muscle strength and muscle strength after 20-minute contact with CO₂ hydrate or ice were measured. For the noncontact group, pre-fatigue muscle strength and post-fatigue (after leaving for 20 minutes after carrying out the fatigue task) muscle strength were measured. Induced muscle strength (induced torque) upon electrical stimulation to the triceps surae muscle was used as an index for muscle strength.

The induced muscle strength was determined by inducing muscle contraction through electrical stimulation given to the triceps surae muscle using a constant-current electrical stimulation apparatus (DS7A and DS7AH, manufactured by Digitimer Ltd.), and measuring the muscle strength in this operation using a myodynamometer (CON-TREX MJ(R), manufactured by Physiomed AG).

The method for giving the electrical stimulation was as described below, and the intensity of the electrical stimulation to be given was determined as follows.

In order to perform electrical stimulation, a negative electrode (disposable earth electrode, manufactured by Gadelius Medical K. K.) was attached proximally to patella, and crocodile clips were put thereon. Before attachment of the electrode, hair around a region proximal to patella was shaved, and the region was wiped with absorbent cotton moistened with an alcohol. A positive electrode (Red Dot(R), manufactured by 3M Japan Ltd.) was attached to the back of the knee, and crocodile clips were put thereon, as in the negative electrode. In order to determine the position of attachment of the positive electrode, each test subject took a standing posture, and moistened absorbent cotton held between crocodile clips was placed on the back of the knee, followed by the flowing of weak current. The current flowed to the nerve so that muscles contracted to move the leg toward the direction of plantar flexion. By exploiting this, a site where the leg was most greatly plantarflexed was found, and the site was determined as the position of attachment of the positive electrode.

After attachment of both the electrodes, the intensity of the electrical stimulation was determined. Each test subject lied in a prone position on a myodynamometer (CON-TREX MJ(R), manufactured by Physiomed AG), and the myodynamometer was fixed such that the center of its axis of rotation fit with ankle joint. In this respect, the angle of the ankle joint was set to 0°. The voltage was elevated in increments of 10 mV from 30 mV so that current intensity was elevated. A torque (muscle strength) was confirmed at each voltage. The current was allowed to flow to muscles while the voltage was elevated in stages until the value of the torque became constant. When the voltage was elevated in increments of 10 mV, a rise in torque value became 0.2 Nm or less. This case was evaluated as a torque value that became constant. A value obtained by multiplying by 1.2 a voltage value before finally elevating the voltage by 10 mV was regarded as the voltage of the electrical stimulation (i.e., the intensity of the electrical stimulation) for use in the experiment. For example, when a rise in torque value by elevating 60 mv to 70 mv was 0.2 Nm or less, 72 mv (60×1.2) was determined as the voltage of the electrical stimulation for use in the experiment. Induced torque by electrical stimulation at rest was confirmed at the determined voltage. The induced torque was obtained by instructing the test subject to be placed at rest, and performing electrical stimulation by twitching (twitch torque) twice in pre measurement and electrical stimulation by strong contraction (triplet torque) twice at an interval of 10 seconds each. Output torque signals were recorded in a personal computer using an A/D converter (PowerLab 16/35, manufactured by ADInstruments) and dedicated software (LabChARt8, manufactured by ADInstruments). Such a method for measuring an induced torque is a noninvasive approach and causes no harm on research targets.

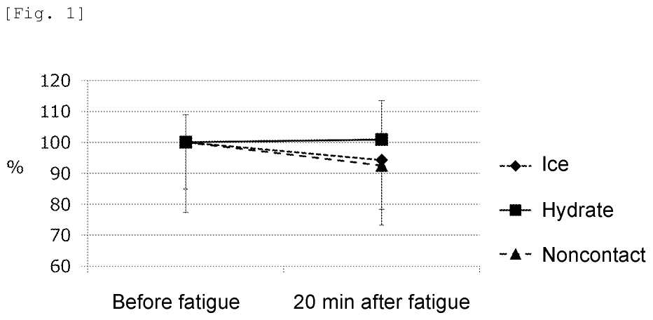

In this way, for the CO₂ hydrate group and the ice group, pre-fatigue (i.e., immediately before carrying out the fatigue task) muscle strength and muscle strength after 20-minute contact with CO₂ hydrate or ice were measured. For the noncontact group, pre-fatigue muscle strength and post-fatigue (after leaving for 20 minutes after carrying out the fatigue task) muscle strength were measured. Average pre-fatigue induced muscle strength of each group was defined as 100%. FIG. 1 shows average induced muscle strength of each group at 20 minutes post-fatigue (for the CO₂ hydrate group, after contacting CO₂ hydrate for 20 minutes; for the ice group, after contacting ice for 20 minutes; and for the noncontact group, after leaving for 20 minutes).

The results of FIG. 1 were subjected to two-way analysis of variance (group [test product group, control product group, noncontact group]: correspondence absent, time [before start of fatigue task, 20 minutes after completion of fatigue task]: correspondence present) targeting the measurement values of induced muscle strength upon electrical stimulation. As a result, significant difference (P value=0.022) was found as to group×time interaction. As a result of also carrying out multiple comparison, the induced muscle strength after ice contact in the ice group, and the post-fatigue induced muscle strength in the noncontact group exhibited significant reduction as compared with the induced muscle strength before the start of the fatigue task, whereas the induced muscle strength after CO₂ hydrate contact in the CO₂ hydrate group exhibited no significant reduction as compared with the induced muscle strength before the start of the fatigue task. These results demonstrated that the contact of CO₂ hydrate or melted water thereof with the skin can promote the recovery of muscle fatigue.

As a result of conducting multiple comparison as to induced muscle strength before the start of the fatigue task in the aforementioned three groups, no simple main effect was confirmed. In short, no significant difference among the groups was confirmed as to the induced muscle strength before the start of the fatigue task.

### INDUSTRIAL APPLICABILITY

The present invention can provide a muscle fatigue recovery-promoting agent that can effectively promote the recovery of muscle fatigue, a method for producing a muscle fatigue recovery-promoting liquid, comprising the step of contacting the muscle fatigue recovery-promoting agent with a liquid, etc.

1. A method of promoting recovery of muscle fatigue in an animal, comprising the step of applying ice having a CO₂-content rate of 3% by weight or more; or a liquid comprising 200 ppm or more of carbonic acid and containing 5 million or more ultrafine bubbles/mL to a systemic or local skin of the animal.

2. The method according to claim 1, wherein the ice having a CO₂-content rate of 3% by weight or more is CO₂ hydrate.

3. The method according to claim 1, wherein the animal is an animal in need of promoting recovery of induced muscle strength upon electrical stimulation after exercise.

4. The method according to claim 1, wherein the ice having a CO₂-content rate of 3% by weight or more is ice having a size of 3 mm or larger in terms of a maximum length and having a CO₂-content rate of 3% by weight or more.

5. The method according to claim 1, wherein the ice having a CO₂-content rate of 3% by weight or more is a consolidated CO₂ hydrate.

6. (canceled)

7. A method for producing a muscle fatigue recovery-promoting liquid for application to a systemic or local skin of an animal, comprising the step of contacting ice having a CO₂-content rate of 3% by weight or more with a liquid or melting the ice as it is.

8. The method according to claim 7, wherein the ice having a CO₂-content rate of 3% by weight or more is CO₂ hydrate.

9. The method according to claim 7, wherein the animal is an animal in need of promoting recovery of induced muscle strength upon electrical stimulation after exercise.

10. The method according to claim 7, wherein the ice having a CO₂-content rate of 3% by weight or more is ice having a size of 3 mm or larger in terms of a maximum length and having a CO₂-content rate of 3% by weight or more.

11. The method according to claim 7, wherein the ice having a CO₂-content rate of 3% by weight or more is a consolidated CO₂ hydrate.

12. The method according to claim 2, wherein the animal is an animal in need of promoting recovery of induced muscle strength upon electrical stimulation after exercise.

13. The method according to claim 2, wherein the ice having a CO₂-content rate of 3% by weight or more is ice having a size of 3 mm or larger in terms of a maximum length and having a CO₂-content rate of 3% by weight or more.

14. The method according to claim 3, wherein the ice having a CO₂-content rate of 3% by weight or more is ice having a size of 3 mm or larger in terms of a maximum length and having a CO₂-content rate of 3% by weight or more.

15. The method according to claim 12, wherein the ice having a CO₂-content rate of 3% by weight or more is ice having a size of 3 mm or larger in terms of a maximum length and having a CO₂-content rate of 3% by weight or more.

16. The method according to claim 2, wherein the ice having a CO₂-content rate of 3% by weight or more is a consolidated CO₂ hydrate.

17. The method according to claim 3, wherein the ice having a CO₂-content rate of 3% by weight or more is a consolidated CO₂ hydrate.

18. The method according to claim 4, wherein the ice having a CO₂-content rate of 3% by weight or more is a consolidated CO₂ hydrate.

19. The method according to claim 12, wherein the ice having a CO₂-content rate of 3% by weight or more is a consolidated CO₂ hydrate.

20. The method according to claim 13, wherein the ice having a CO₂-content rate of 3% by weight or more is a consolidated CO₂ hydrate.

21. The method according to claim 14, wherein the ice having a CO₂-content rate of 3% by weight or more is a consolidated CO₂ hydrate.
